SEQUENCE LISTING (27 sequences)

SEQ ID NO: 1 - DNA, 849 bp, Artificial Sequence
  synthetic
atggatcaagatgactggttgggaacgctaagatacgccggtaaggcgcaggataaagtttccgttgacgctctcatgctccggtaccgtccgatcgctccaaagccgacgactggtcagccatgtggtgtggcagataacaacaacaatagctcatacggtatgagcaaacgaaccaaacgaaagtacgttagggtttcgaagaataataaaggcacgtgtcgaggtaagagcagatctgacttgtctgatgatcgggaacaaactgatgtcgtgacgttgcaactcttgccggaaaaatcagatatttccggtgagtactcgccgttggatcaagatagtctcgatccgtcggtgaaatcgataatcggagaggaaacacaagaaaccaacacgtggggtatgtttaacggcagcgtcacggcggagatggagacgtgggtgacggtggagtccgtcacaagcgtgtgtgagggtagtttgagttcccatgcggtggggattacggacgttgagatcgttgataatcttggtaaggacacgtgtccagcgttcgtatcggatggctcgaaccgtgtggtgtgggtcaacgaggcttaccggagaaatgtttccggtgatgattcaacggcgtcagtatcgccggaagttgtggtgtggttggtggcggaggaggcaacggcggcgatgcattgtaactaccaagctttcacgtgtagggtgaggatgcaatacacgtggaaggaaacaaagtataccaaaacggtgccgtgtgatgtgtggaaaatggagtttggtggttttgcatggaggctagacacaacagctgctctgactctctggctttga

SEQ ID NO: 2 - DNA, 885 bp, Artificial Sequence
  synthetic
atggacggtaggggagggtgttgcatagctaggtacgccattggctctggaccgtacgatctttccaaagcagatcgcatcatgcttcgatttcgtcccattgctcctaaaccggccagccccggcggagtaaatcccgtatccagcggagatagcggcggcggaagctccgacgtttcctttagatcagctagtagaagaaagaggaaatgtcatcagctgaaggagaatggtggtaacgccaagaggtgcacccggcggaagacgtcggataaacccgtcgttcacggcagtgctaatgcggtgactctttctctattaccggagaaacctatagatcttaaggcggcggtggagaagcagaagcggcagggtccgttatggttgagtttcaaagacggcggagggatgctcacaccggcttatcagacaccggagattgtgcagaggacggtggtgatttcgtcgtgtatgacggtggagcgtgtaacggacgcttggattgacggttacggtttagggaggtcagatgaagaaaggaagatgaatcttgtgagagacacgtgtcctggtttcatatccgacggttcagggagagtcacgtggactaatgacgcgtatcggaagatggctagggatattattcccgtggaagaaggtgcaccggagataactagcggcgatagttttcacgtgatcgtacggttggtgatgagggagaggccgatgctaacgtcccctggattcacatgcagaatgaaactccagtacacgtgtcaaaatcgtgagagaggctcagtcacggtgccttgcgacgtgtggagaatggacgtcggtggtggttttgcttggaggctcgacgttaaggccgctttgtgcctttga

SEQ ID NO: 3 - DNA, 912 bp, Artificial Sequence
  synthetic
atgcatggcaacggaaagcagtgtcaagcttctatttgcgacggtgcaacagatcaggacaagaccgttataagcaagataatgcaacggttccgtccgatcgcaccaaaaccagctgtcggcgaatcttcagacgatacaaatagtgatagattccttggaagaaacagaagatcgaaacgaaagtatgttagggttcgggacaagaaaaatagtagtggtagtaataataagaaggatattactggtaaaaagaatggttgcgatagagggaacataaagaccgatcttgataaaaagatagacggtgatgatagaaccgatatcgtcacgcttcagctgctgccagagaaggatagagatataggaaacaatggagataaagctggtgagttttgttcggatctgagtgatatggatccaaaaaagagcttgtataattcgatcatagggctatcgtcatcttttgatcggaaggtggtggagtcgtggttgacggtggagtgtgtgagtgacacgtgtactgacttgggatggtatcacatcctggagcagctaggccgcatggatcaggcagaggagagagtgatgaggatgttagaggttgacacgtgtccatggttagtgtcggatgggtcaaaccgggtctgttgggtaaaccgggcctaccggaggatgatgggagctcccgacgtggatgttatcagggtgtggctggtggtggccatggaccttatggaggagatagcatgcatggtggagttatacggtgcggtgacgtgcagggttagggttaggtacgagccgtccacgtggaggaagatgacggtgccgtgtgatgtatggaggataagatcaggtgggtttgcttggagattggatgttgaatcagctctaaggcttggcatgtga

SEQ ID NO: 4 - DNA, 819 bp, Artificial Sequence
  synthetic
atggatcaagatcatgagttatggcggacgctaagatgcgccggtaaagcgcaagataagacctctgttgatactctcatgcttaagtaccgtccgatcgctccaaagccgacgactactggtcaaccattggtcggagatacaagtagcacgagaaggacgaagagaaagtacgttagggtttcaaagaataataaagccacgtgtcgatcaaagacgaacggcttcagatctagctcaacagatccggagaatggtcgggaagatatcgtcacgctacagctcttaccggagagatctacgccgttgagtttagatcataataatctcgatccaacggtggaaacgataaacggagatgaaacttgtaataccgacacgtggctcaagtttaacggcggtgatgacgcgttgcaacaagttccggtagagacgtgggtgacggtggagtccgttaacagtggcttggtttcccatgcggtagggttaacggacgaagagctcacatatgctttagataaagacacgtgtcctggtttcatatcggatggttcgaaccgtgtggtgatggttaacgaggcttaccggaggatagtaaccggagacggcggattcggaagagaggtgatagtgtggttggtggttgaccaaacggcgacgttttgtgactaccgaacttttacgtgtaaggtgagaatggaatacacgtggcgagaaacgaagtacactaaaacgttgccgtgtgatgtgtggaagatggagtttggtggatttgcatggaggttggatactactgcagctttaactctttggctttga

SEQ ID NO: 5 - DNA, 876 bp, Artificial Sequence
  synthetic
atgaatttccggggaggttgttgtattgcgaggtacggtggtagtggtggtggaaatgatatgtcgaaggttgatcggattatgctccggtaccgtccgattgcaccgagacctgattctggtggatctcctgcttcaccgacggagaagaatggatcggtgataactaacgtttcgtcgagatcacggagaggtaagagaaaatattcaaaggagaataatagtagcagtaccggatccgttaatagtaacggtaacagcaaacgacagagaaacgatgagacgaagaacggatctggtggtggtagagagatcgtgacgttgcctcttcttccagagactcctgagaagaaagactcgccgttgaaggctaaggcggcgccggagttaggtgcggcggcgttgtggctgagttttaatgacggagctagttataacagacgttatcagacagagttaatgacggaaacggttgtgtcgtcgttgttgacggtggagtgtgtaacggagagattgatggagggagagtatgagttagggtgtacggacgaggagaggaagatgaatcttgagagagacacgtgtcctggttttatatcggacggtttagggagagtcatttggacgaacggttcttacagagagttggtggttgggaaagatcatgagcagtgtagtaagatgagtgtgtggcttgtgatgaaggagaagcctttggtaacgtaccggacttttacgtgtagaatgagattgcagtacacgtgtcgtgataaggaggtgagttcgattacttcgttctgtgatgtttggaggatgagtgacggtggctttgcgtggcggcttgatgttgatgctgcactttgccttggacggtga

SEQ ID NO: 6 - DNA, 909 bp, Artificial Sequence
  synthetic
atggacggtaggggaggttgttgcatagccagatatggtggttacggtggtcgctacggtctctccaaagcagatcgaatcatgcttcgttttcgtcccattgctcctaaaccggccagcgacggtggaagtgtctctctcaccgggaagagcggctccaccacaactacaagtggtggaagctccgatctctcaggtaaatctgggagaggaaagagaaagtatcaaaaagattgttccggtgggaactcccgaaggtgcaataagaagaagagagatctaagtggagacactgctactaccacggcggttacattgtctcttttaccagagacgcctgagaaacgagtttttccagatctgaatgcttttccggtggagaagcagaagcgaaacggtcctttatggctgagtttcaacggcggtggtgagattttaacgccgtacaaaacggcggagatatcacgaaggacggtggtggtttcgtcgtgtgtgacggttgagcgtgtgactgacgcttggatcgacggttatggattaggggagacaaatcaggagaggaagatgaatctagtggaagacacgtgtcctggttttatatcggacggtgtagggagagtcacgtggaccaatgaggcgtataagaagatggctagagaagatattaatattccgatggaggaaggtgtaccggaggatattagttacgataattttcacgtgaacgtacggttagtgatgaaggagaggccgatgctaacgtacccagctttcacatgcagagtgagattacagtacacgtgtcaagatcgagaaagaggatccgtcacggtgccttgtgacgtgtggagaatggacggcggtggttttgcgtggaggcttgacgttaaggcagctttgtgtctgtga

SEQ ID NO: 7 - DNA, 843 bp, Artificial Sequence
  synthetic
atggacggtggaaatggttttccagcagctaagtacgccggtggaaaaaacgataccacgataatgaaccggataatgctaagattccgtccgatcgcaccgaaacctgtcgccggaggttcatcggatggatctacaccggaaaataacaacatggaacttgttaccaaaagaagagcgaagagaaagtacgttagagttaagaaaagtagcaagtgcaagtctaacaaagaagacgaagcaaataaagacggatcttcattgtatgaggataacggagctggtataactctacagctgatgccgcaaagcagtagcggcgtccgaaatagtctagaaaattctggatctcgacctttctggatgaattttcagaaatcggagaacagtgagatttttcccgtaggatcagatcagatagatcggacggttgagatgcagaagaaaagagtggtggagtcatgggtgatggtagaccagataacaaacgcgttggtagacggagaagcgttaggtagtacggatacagagaagatgaagaatctggaggctgacacgtgtccagggttgatatcagacggtttagatagagttcggtgggtgaatctggcgtaccggagaatggttgatcctctagaaggttccggaaattcgccggagctggtgacgtggctggtagtgaaggagaaaatattgttgcctaactcatcagcttttgcatgcactgtgaggatactgtacaccaaaaactcacagacaatgccatgtgatgtatggaagatggaatttggaggatttgcatggagacttgatgctaaagctgctcttagattgggtcgttaa

SEQ ID NO: 8 - DNA, 828 bp, Artificial Sequence
  synthetic
atggaccttgcaaatacctgggccgggccgggatacacacctacacccaaagataccaccctaatcaaccagatgatgctcaggttccggcctattgcgcccaagcccgtagataattcttctggcccgccggaaactcatgtggttgcaaacagaagaaccaagagaaagtacgttagagttaagaaaaataagaataataaaaaagaaaaaagtgatggattgctggatgaggtggtaactcttcagctactgccggaaagtagcggaggagttaaaacttcgccggaggacagatcctacccaaaaactatcaactttttggtgcaattagaccgatctatatggataaataaaaatattttatcgatcggtgcaccagatccgtccgtagagatccgatctccgatggttgtggaatcatgggtgacggtggacggtttaacaaacactacttttgtagatttatcagctttaggaagtacggacatggagaagatgatgaatctacagcgtgacacgtgtccagggttcatatcggacggtttagatagcgtgaagtgggtgaatctggcgtaccggagaatgatagatccggaagaggagggaggagaagcgacggagatggtggtgcggttagtagtgaaggaggataagcgtgcaccagtattattattattgccaagttttgcatgcatagtaagaattgtttatacatggaataaagtgaagcagtcaaggacaatgccttgtgatgtatggaagatggactgtgggggatttgcatggaaatttgatgctaaggctgcacttagcttgggccgttga

SEQ ID NO: 9 - DNA, 933 bp, Artificial Sequence
  synthetic
atggatgggagaggagggtgttgtattgctaggtatgcaggaggtgcgtacgatatgtcaaaagtggataggataatgcttaaattcagacctatagcacctaaaccagcagcagccggtacttccgtttccggcacgactactccaccgcaaaaatctgaggttccggttcgtactggacggtggaagagaaggtatgttaaggacaataaaaatagtaataataacaagagatctagtagcggcgggtgtagtcctactcctagtcgtaggaaaaggaaagctcggtcggttgaagaaaatgattcaagcgcaaaatcagtgtctggcggagaaacggcagttactctgccgttgttatcggagattcctgaaaggaaggataattcagttgatatgatgaaaaaaaatgctccgatctggttgagttttggtggtaaccaagggaataataatgataacagtaataataataatggtcagttgcagggagtggctatggatcggtcagttgtgatgtttcctcagccagtacgtgtagtggggtcatgggtaaaggtggaaagcgtgacagacgcgtgggtggaagggtatgggttaggacgtacggatgaggaaaaacttataaatctggagcgggacagctgtccagggtttatatcagacggtttaaatagagttaggtgggccaacaaggcatacaaggggatggtgggtgatggggcaggggaagtggttgtttggctagtgatgaaagatgctgtacggctgccggagagcaaatctacggcggcgttcacttgccgggttagggttgtcaggtgcgggaaggaaaaaaattcattgattctgccgtgcgatgtgtggagaatggacggcggaggatttgcatggaggttggatacagaagccgctctctctttggggcggtag

SEQ ID NO: 10 - DNA, 1083 bp, Artificial Sequence
  synthetic
atgatgcagactttgaatccttacccttcaacctcaaaaacagctgagatcatggctagatataggccaatagcaccaaaacctgaggctccaactagtcctgtttctgaggataatccaactggattgcccccaaatattcaaaagtctcctttcttgaggaatgtatggcctcaattgcaagcaaggccaacaaggacaaggaagagaggaaggactgcacttggtccaccttctatgaaaagagccagaggtaattactttcctgctggccagtttcctaattatcatcaggtggtggctgcttctccttcttatagaccaaatgtggttccccagttcactttaatcccaaatcttctccctcttaaatgtggcttgggtacttctgttacaactccatcaaattccataacactccctcttatggcttgtactaccactactcttcctatgctagtggagaaacactctggagaggaaattagagggatcgatttgaatttggcagctgatggccctgaagaattagatttcatgccccaattgcaaggtcctaagacccctggcccggttgttataaccccgcagccagtgcgcccagttgggtcaagtatttccattggttgtatcaatgaggaggaggctccagatggtggtgcaacaaacaagaaatttataaagaaaccagaggaggtggaggaagaagttgaagcagaggctttgccagctgtcgtttcggactcgaataacaaagtaaggctcacaaatgcagcatacaaagagatggtgggtcaacctgagtgttgctggttggactacatggttgggaatgcatgcaagagaattggtggggaagtgatacttgagttcttggattcatcttgcagtgtgccaatgtcatctgatggattcaattgctgggtgaagatagaatggggagctgcacaagggaaaaagaattcagtcaaagctttctgcaatgctgtgaagttagcttgccaatccaaagattatgtctttgagtggaggttccataccacagatgataatactcctgaatcagctgcttccaatatttaa

SEQ ID NO: 11 - DNA, 747 bp, Artificial Sequence
  synthetic
atgtccaagatggacaaaataatgttacgctttcgccctatcgcccccaaacccctccccgccgccgccgccgctctttccgacgcttcctcgtcggaaagcaccggctccgccaagcggaaggataacaccgccagcaagcggtgcagtcgagggatccgccggaggagaaacgcgcctcctcctcctccttctccggcggtgactctcccgctgctgccgggatctccaggtccgaagaaaatcacctcagaactaaagaacaagaatgtacctgtgtggctgagcttcgagaacaacttcgagaaccgcggcggtgctgcttcggagaagctggatccttgctggtactcgcaggcgacggcggcagcgggatcggtggtgacggtggagtgcgtgatggacacgtggcagcagcaggatgaggggttagggttggggagtggagacgaggaacggaaggtgaagctgaaagaggacacgtgtccggggtttatatcggatggttacgggagggtgacgtggacaaatgaggcgtacagggaaacagtaggtgctggaggagtatggttggcaatgaaagtggccgtaccgtatccgtacagaggtttcacgtgcagggtgagggtgcggtacgcgtgcgggatagaaagaacggtgccatgtgatgtgtggagaatggacagtggtggctttgcctggagattagacgttaaagctgctcttagcttaagcttagctttctaa

SEQ ID NO: 12 - DNA, 870 bp, Artificial Sequence
  synthetic
atggatttaagaggaggctgttgcatcgccaggtatgtacccggtgcatcccacgtgtccaccgtcgacaaaataatgcttaggtttcgtccaatcgctcccaaacccgccgccggagccaccgcttccgacggatcttcgtctgaaaccagcgacgcttttctcagaaacggaaacacgaagagaaagtacgtaagagatagcaactacaccagcaaacgtagaatctgccggagaaagaactctaattcctcaccggagcagaagcagaagcaaacgacgccggctgtgactcttcctttgcttccagaaactcccgatcggaaggattttccggcaaaggatctaactccatcaccagtgcgaaacaacaacaacaacaacagcagcaacaagagcaagaagttgaatattattaacaagaacgtgccggtgtgggtgagttttgcgaatcgaagccttacgatgatgggagggtggtgttcgtgtgtgacggtggagagcttgacggacacgtgggtggagggcgagtggctggggagtacggacgaggaacgcagagtgaatctgagcaaggacacgtgtccggggttcatatcagacggttacgggagggtgacggggacgaatgaagcatacgagaagatgatggagggtgatgagggacagggaccagtgttgctggtgaataaggtgaacacagtggttccgcacgcgtcgttcacgtgcttggtgagggtggtgcaatacgcgtgtgggagggagagaagctctctcacggtgccctgcgacgtgtggagaatggactctggtgggtttgcatggagattagacgttgaaactgcgcttagcttgagactgggttactaa

SEQ ID NO: 13 - DNA, 924 bp, Artificial Sequence
  synthetic
atggacggtgcagacgcgtggcgcgtggccgcgtgtgccaacaccaacgtcaacagaataatgctccgctttcgcccgatcgcgccgaaaccggtcgccggaagttccgccgtctccagagccaccggcgctggcgacggaagccagagtagccacgtgtcagttctagggaagagaccgaagaggaagtacgttaggattcggaggaacggtggatacgtgcgaaagaataacggaaacagtaaccgaaaaagtaattgtaactgtaacgatgagtcttccgacgtggcggttgtgacgttgcagttgatgccggagaaggacgcgccggaaggagacgtcactctcgcgggagattcgtggtgtaagaacgttgatcttgacctaactgtggagaagattcagatcgtagagaatcggagtgttccgccgccacgcttggtggtggaggaaggcgagggtgccaagggttcagatcttgttccggcggcgaaggcggcggagtcgtgggtgacggtggagagcgtgacaggcacgtgcatgggggagggagagggagggagagggttattaagttgtacggacgaggagagggtgaagagcctagaaactgacacgtgtccggggtttgtctgtgacggaagccttagggtgcggtgggtgaatgatgcgtacaagaggatggtgctggaggggcgcaagggtgagggtgaggatatcatggtgtggctgaaggtgaaggatagtgcctgcgctgcgtggtggtgctattctcacccagcgttcacctgcggggtgaggctgcagtacacgtggcggaacgagaagtgcacgaagatggtgccgtgtgatgtttggagattggattgtggcgggtttgcatggaggctagacgtgaaagctgcgctcagtttgggactctga

SEQ ID NO: 14 - DNA, 741 bp, Artificial Sequence
  synthetic
atgtccaagatcgatcaaataatgttacgctttcgccctatcgcccccaaacccctcccctccgccgccgccctttccgacgctttctcctcggaaaacaccggcgtctccaagcggaaggataacactggcagcaagcggtgcagtcgagggatccgccggaggagaaacgcgcctcctcctcctcctcctccgcctccggcggtgactctcccgctgctaccggaaactccagatccgaagaaaactacctccgaactaaagaacaagaacgtaccggtgtggctgagcttcgaaaacagcttcaagaaccgcggcggtactgcttctgagaaggtggatccgtgctggtactctccggcagcgggatcggtggtgacggtggagtgcgtgatggacaggtggcagcctcaggaggagggactagggttggggagaggagacgtggaacggaaggttaagcttgaggaggacacgtgtccggggtttatatcggacggttacgggagggtgacgtggacgaatggggcgtacagggaaattatgggagagggaggagtatggttggcaatgaaggtgaccgtaccgtgtttgtacagaggtttcacgtgcagggtgagggtgcagtacgcctgtggtaaagagagaacggtgccatgtgatgtttggagaatgaacagtggaggctttgcctggagattagacgttaaagctgctcttagcttaagcttagctctctaa

SEQ ID NO: 15 - DNA, 873 bp, Artificial Sequence
  synthetic
atggatggaagaggaggctgttgcatcgccaggtatgtacccggtgcatcccacgggtccaccgtcgacaaaataatgcttaggtttcgtccaatcgctcccaaacccgccgccgcggccaccgcttccgacggctcttcgtccgagagcagtgacgcttttctcaaaaacggaaccactaaaagaaagtacgtaagagataacaactacaccagcaaacgaagaatccgccggagaaagaacacttcctcgccggagaaaaagcagaagccggtggtgactcttccgttgcttccggaaactcccgatcggaaggattctcgggcaaaggatctaactccatcaccagctaaaccagtacgaaacaacaacaacaacaacaacaagagcaagaacttgaatattattaacaagaacgtgccggtgtgggtgagttttgagaatcgaagccttacgatgatgggagggtggtgttcgtgtgtgacggtggagggcgtgacggacacgtgcttggagagcgagtggctggggagtacagacgaggaacggagactgaacctcagcaatgacacgtgtccagggttcatatcagacggttacgggagggtgacgtggacgaacgaggcgtacgggaagatgatgatgatgaagggcgagggtgatgagggacagggaccagtgttgctggtgaataaggtgaatacggtggttccgcacgcgtcgttcacgtgcttggtgagggtggtgcaatactcgtgtgggaaggagagaaactcgctcacggtgccctgcgatgtgtggagaatggactgtggtgggtttgcatggagattagacgttaaaactgcgcttagcttgagattgggttattag

SEQ ID NO: 16 - DNA, 930 bp, Artificial Sequence
  synthetic
atggacagtgcagacgcgtggcgcgtggccgcgtgtggcaactccaacgtcaacaaaataatgctccgcttccgcccgatcgcgccgaagccggtcaccggaatttccgccgccgaagctggctccgccgtcggagaagttactcagaacaaccacgtgtcagtcctggggaagagaccgaagaggaagtacgttaggattcggaggaacagtggatacgtgagaaagaataacggagacagtaacggaaaaagtaacggtaacggtaacgatgagtcttccgacgtggcggttgtgacgttgcagttgatgcctgagaaggacgcgccgggaggagacgtcacgctcgccggagattcgtggtgtaagaacgttgatcttgacctaaccgtggagaagattcagatcgtggagaatcggagtgctccaccgccgcgcgcggcggcggaggaaggcgagggaggtaagcgttcagatcttgttgcggcggcgaaggtggcggagtcttgggtgacagtggagagcgtgacaggcacgtgcatgggggagggagacggagagggagggcgagggttattagattgtacggacgcggagaaggtgaagagcctagaagctgacacgtgtcctgcttttgtctgtgacggaagccttagggtgcggtgggtgaatgacgcgtacaagaggatgatgctggaggggcgtgagggtgagggtgagaatatcgtggtgtggttgaaagtgaaggatagtgccatcgcttcgtggtggtgctactctcacccggcgttcacctgcggtgtgaggctgcagtacacgtggcggaacgagaagtgcacgaagatggtgccgtgtgatgtttggagattggactgtggcgggtttgcatggaggctagacgtgaaagctgcgctcagtttgggtctctga

SEQ ID NO: 17 - DNA, 933 bp, Artificial Sequence
  synthetic
atgaagacagactacggaaagcagcgggaagcttctgcttgcgatgatgttacggatcaagacaagaccgttgtaagcaggataatgcaacggttccgtccaatcgcaccaaaaccacctgtcggcgaatcttctgacgacacaaagagttgcagattcctccgaagaaaccgaagatttaaacgaaagtatgttagggttcggaataaaaacattagtagtggtagaagtaataataataaagatatgatatgtagtaataagaaagcttgcggcgatggagagaagataaagacggatcttgatgaagagataggcggtgaggataagagcgatatcgtcacacttcagttgcttccggataaggataaagatcatcaagctggtaaggataatacggatccgagtgatatggatccaagaacgagtttaaatatatcatcatctttagatcggacggtggtggagtcgtggttgacggtagagtgtgttaataacacatgtactgaactgggagggtatcacaagctacagctaagccgtatgaatgagggggaggagatagtgatgaggatgctgtattttgacacgtgtccttggttagtttctgatggatcaaaccgggtctattggatcaaccgggcgtaccggaatatgttgggagatccgaaggcagaggtgactatcaaagtatggctggtggtggcgacggaccttatggaagagatgtcgtgcatggtggagtcgtacggtgcggtaacatgcagggttagggttcggtatgatacgtcgacgtggaggaatgagaagagtcactcggagtcagaagtgaagatgactgtgccgtgtgatgtatggaggatgggatcatcaggtgggttggcttggagattagatgttgaatcagctctaacacttggaaggtataactga

SEQ ID NO: 18 - DNA, 834 bp, Artificial Sequence
  synthetic
atggatcaagatcacgagttatggaggacgctaagatgcgccagtaaagcacaagataagacttctgttgatactctcatgcttaagtaccgtccgatcgctccaaagccgacgaccactggtcaaccatttgtcggaagcacgagcacgacaagaacgaagagaaagtacgttagggtttctaaaaacaataaagccacgtgtcgatcaaaggacgacgttagatctagctcaacagatccagagaatggtcgggaagatctcgtgacgctacagctcatgccggagagatctacaccgttgagtttggatcttaataattttgatccaacggtaaaaacaataatcggagatgaaacctgtagaaccggaacgtggctaaagttcaacggcggagaagacgcagtgcatcaggttccggtggagacgtgggtgacggtggagtccgtaaacggtggctccgcttcccatgcggtagggtgtacggacgaggagattgcggaggctatagataaagacacgcgtcctgctttcatatcggacggttcgaaccgtgtggtgatggtgaacgaatcttaccggaagactgtaaccggagactgtgggtttggaggagagccgtcgtcgttgttgccggaagttattgtgtggttggtggttgaccaaacggccacgttttacgagtacagaactttcacgtgtaaggtgaggcttgaatacacgtggcgagagacgaagtatgttaaaacggtgccgtgtgatgtgtggaagatggagtttggtggatttgcatggaggctagacaccactgcagctttaactctttggctgtga

SEQ ID NO: 19 - DNA, 900 bp, Artificial Sequence
  synthetic
atggacagtaggggagggtgttgcatagcaaggtacgccggtggaggtggaacgtaccattacgatctgtctaaagcagatcgcatcatgcttcggtttcgtcccatagctcccaaaccaactagcgacggcagcggaggaaagcccgtatccagcggagagagcggtggcggaagctctgatgtttcctttaaaggaggaagaagaaagaggaaatgtcagcagaaagagaatagtggtaacgccaggaggtgcactccgcggagaagatcagataaatccgtcggtcacggcggacatactaaggtgacactttctctgttacctgagacgccggatgaaggtgattttacagatcttaaagtgtcggtgccgtcggtggagaagcagcacggtccattttggctgagtttcagcgacggcggtgggatgtttactccggcttaccagacggatgttatgtggaggaaggtggtgatttcttcttgtatgacggtggagcgtgtatcggacgcctggatggacggctatggtctagggaggtctgacgaagaaaggaagatgaatcttgtgagagacacgtgtcctggtttcatatcggacggttcaggtagagtgacgtggacaaacgacgcttacagaaagatggcgagggataatattccggaggaagatggttcaccggagaatatgagcggcgatagttttcaagttatcgtacggttggtgatgaaggataggccgatgctaacgtacccggctttcacatgcagggtgaaactacagttcacgtgtcaagatcgtgagagaagctccgtcacggtgccttgcgacgcttggaagatggacaatggaggtttcgcttggaggcttgacgttaaggccgctttgtgcctttga

SEQ ID NO: 20 - DNA, 1347 bp, Artificial Sequence
  synthetic
atggacgttagaggaggatgttgcatagcaagatatggtggttacggtggtcactacagtctctccaaagcggagcgaatcatgcttcggttccgaccaattgctcccaaaccatccaacgacggcggcggtggatctcccggcgccggaaagtattgctcctctacaacaagcggtggcagctccgatgtatccagtaacacaaagagaggaaagaggaagtatcataaggagagttcaggcgttaactcccggaggtgcaacaagaggaagagaccgaacactagaacggctgttaccttgtctcttttaccagatctgaatgtatctccggtggagaagcagaggcaaaacggtccattatggctaagcttcagcggcggagaccatgagatgctaacaccgtacaagacggcggagatctcgcaaagaacggtggtggtctcgtcgtgtgtgacggttgagcgtgtgaccgacgcgtggactaacggttatggtttagggaagaccgacgacgagaagaagttgaatctagcgagagaggcttgtccagggtttatatcggacggtgttgggagagtcacgtggaccaacaaggcgtataggaagatggcgaaggaagatattaatattccggtggaggacggtgcaccggggatgagttatgataattttcacgtgatcgtacggttggtgatgaaggagaggcccatgctcacgtacccggcgttcacatgcagcgtgaggctacagtatacgtgtcaagatcgtgagagaggttcagtcactgtgccttgtgatgtgtggaggatggacgatggaggttttgcgtggaagcttgacgttaagaccgctttttctcttcaccaaattatttacttaatagtgtgccatagcaaatataacattctttacgatcacaagcccataccaaaaatcacggaccacaaaatcaaaagtaagtaccacaagatctattcaacactttgtatcagtccgaagttcaactttaaactcttgtaccccataacaaaattaacatccaagttcattcgaggtttaaaaaaagatgctgaaacctcaagaatctgcagtaaagaaaagatgctggaaacaagtggacaatcacagcaggaacatgtagaagttggcaaagttatagattatgtacatatatatagtccattctctggttcacaaagcatccattggcttgcaagatttacataccatcgatttttaagcgcaaaacgacgacgttctgatggtaaatactttgtatctgacctaaacgaagtcgttttagtttcgaaatatttgtttatctgccgaacgacgccgttttaa

SEQ ID NO: 21 - DNA, 846 bp, Artificial Sequence
  synthetic
atgaatcaagatgagtggttgggaacgctaagatacgccggtaaggcgcaggataaggtttcccttgacactctcatgctccgttaccgtccgatcgctccaaaaccgacgactggtcagccatgcgatgcaggagacaacaacaagaactcttacaatctgagcaaacgaaccaaacgaaagtacgttagggtctctaagaataacagcaccacgtgtcgaggaaagaccagatctgacttgcctgatgatcgggaaccaactggagtagtgacacttcaactcatgccggaaaaatccgatcttacaagcgactgcacgccgtcagatccgatggtgaaaacgataaccggagaggaaacacgagaaaccaacacgtggaccatgtttaacggaggcggcacggcggagggggagacgtgggtggagacgcgggtgacggtggagtccgttaccggcgtatgtgacggtagttcgagtttccatggggtgaagtgtacggacgttgagatggtggataacctaagtaaggacacgtgtccagcgttcgtatcggatgcctcaaaccgggtggtttgggtcaacgaggcttaccggagaaacgtttccggtgaagatccgtcatcgttgtcgtcgttgccggatgtcatggtgtggttggtgatggaggagtcgacgatggcgatgtattgtaactacagagctttcacgtgtagggtgaggatgcagtacacgtggcaggaggcaaactacaccaaaacggtgccgtgcgacgtgtggaaaatggagtttggtggctttgcatggaggctagacacaacagctgctctaactctctggctttga

SEQ ID NO: 22 - DNA, 849 bp, Artificial Sequence
  synthetic
atggaacaagatgagtggttgaatacgctaagatacgccggcaaggtgcaggataaggtttccgtcgacagtctcatgctccggtaccgtccgatcgctccaaagccgacgactggtcaaccatgcggtacaggagacaacaataacaacaacaactcatacggtaaacgaaccaaacgaaagtacgttagggtttcgaagaataataaaaccacgtgtcgaggaaagagcagatctgacgtgtctgatggtcgggaacaaactggtgtcgtgacgctgcagctcatgccggaaaaatccgatccgtcaggcgactacacgccggtagatcaggacagtctcgatccgtcgctgaaaacgataatcggagtagaaacactagaaaacagcacgtgggccacgttcaacggcggcgggacggcggaggtggagacatgggtgacggtggagtccgttacaggcgaatatgacggcagttcgacttcacatgcggtggaatgtacggacgtcgagatgatggataatctaggaaaggacacgtgtcccgcgttcgtatcggatgcctcgaaccgcgtggtttgggttaacgaagcttaccggagaaatgtctccggtgaagattggtcgtcatctttgtcgttgccggatgttatggtgtggttggtggcggaggagtctacggtggctatgtattgtaactaccgagctttcacgtgtagggtgaggatgcagtacacctggcaggagacaaagtataccaaaacggtgccgtgtgatgtatggaaaatggagtttggcggctttgcatggaggcttgacacaacagctgctctgactctctggctttga

SEQ ID NO: 23 - DNA, 900 bp, Artificial Sequence
  synthetic
atggagggcggagatcaatggaggatgctaaaatgcgctgggggtgtacaagataagacgataatcaaccggataatgctcagattcaggccgattgctccaaaaccggtgaccggagactcagcttccggtgagtcgacgtttggtaacaagaactcgggtgttacaagcatgagagtgaagagaaaatacgttagggtttgtgagaagaataataggagaagaagagccttggatgaagctaaagacgataaggatggaaataagggttccgtaactctgcagttgatgcctgagagagctgatctagagaaatcagcggttgttgatagctcaggggcgggtgatcatgatctcgatcggacggttggtgatgattatcatcaatctcatgaggacccgccgccgctgtgcttgaagttgcagaagatggttgcaacagatcaagaagtggcggcggcggtgatggggttgtcggatcaggcgacggggaggaaagtgagtgttgtggagtcatgggtgacggtggagagcataacagacagctgcatggatgaaggagacatggggagctgtacggacgtggatagaatgagtaatctggataaggacacgtatccagggttcatatcggacggttggcaccgtgttttgtgggtgaacgaggcgttcaagagcatggtgggcggagaaggggctgaaaccgcggtggggttggtggtgaaagaagggtttaaattcccacagggtgcattctcatgccgggtagggttacggtacggagatggaaagggaaagaaaaagcaatcgaaaatggttccttgcgatgtgtggaagatgagctttggtggatttgcatggaggctcgatgttaaggctgctctcggtttgggactctaa

SEQ ID NO: 24 - DNA, 903 bp, Artificial Sequence
  synthetic
atgaacggtgaagagaagcggagcagggttagctacgcaggtggtgcaccagataagatggtgataaacccgataatgttaagattccggccgatagcccctaaaccggtttccggtgagtcaggttctgatggggttcggtttgataacaaaaatttgttgctttgtaatccaagagctaaaagaaagtacgttagggttcgaaagaataatatgagaagaaagaaaagatcatcgtcagatcaagctcatcatgatgaagcttcaaacaatcctcagacgattgttactttgcagctattgcctgagaaaactgaagggaacggatcgattaacaatcaaaacggtagagttttgggggaaaatcaagatccactttatttatgtaacttgaacttcaataaccgttgggttgatcgtttgggagctgtggaagaacctaatcggacggttctgatgagtcaaacgaggaaagcaacggtggtggagtcgtgggtgacggtggagagcgtgacggacagttgcatggaagtgagacagctagggagtacggacgtggagatgataaagaatctggagagtgacacgtgtccagggtttatatcagatggtttgaatagggttcagtgggttaatggagcttacaagaggatgttgatggtagtaggggaagcaaatgacggccggccgccaccggagataatggtctggcttgtgttcaaacaagaattgcctaggttttgcactgcattttcatgcaaggtaaggttgcagtatacatggcacaaggagaagttctcaaggatgttgccatgtgatgtctggaaaatggaaggcagtggtggtggtggttgtttagcgtggcggctggacgtcgaggctgctctcagtctcggccggtga

SEQ ID NO: 25 - DNA, 900 bp, Artificial Sequence
  synthetic
atgaacagtgatgagaaatggaggtatgttacctacacatggggttcgcaagagaaaacggtgaacatcaatccgataatgctaagattccgtccaatcgctccaaaaccggtgaccggagagtctggttccggtggggctcagtttggtaacaaaaacttgttgctttgtaagccaagagctaaaagaaagtacgttagggttcgaaagaataataccaagagaaagaaaagatcatcatcatcatcatcaccatcagatcatgaagaagcttcaaacaaaactgaaaaggctgttactttgcagttattaccagagaaaactgaagcaattggatcaattaacgatgaaaacggtgtagttttggaggaaaataatcaagatctaccttcgttgtttaacttgaacaacagctgttggattaatcgtatcgctcctttggaggagcctgatcggaaggcggtgatgagtcaaacggaaagggcaacggtggttgagtcgtgggttacggtggagtgcgtgacggagacttgcatggatgggagagagctaggaagtactgacgtcgataagatgaagaatcttgaggccgacacgtgtccagggtttatatccgacggtttaaatagggttcagtgggttaacggggcatataagaggatgttgatggcgggggaagggcgtgaggagtggttgccgccggagataacggtttggcttgtgattaaacaagagttgcctacgttttgcactgcattttcatgcaaggtaaggttgcagttcatgtgggggaacaagtgttccacaataatgttgccgtgtgatgtctggaaaatggacggcggcggtggttttgcatggcggctcgatgtcgaggctgctctcagtctcggccgttaa

SEQ ID NO: 26 - DNA, 603 bp, Artificial Sequence
  synthetic
aaagctgatctaaataaatcaatcgttgttgaaagatcatgggatgttgttgatgatgatgatctggatcgagctttaggtcgtgacaattatcgttttcaagatccaccatcgctgtgcttgaagttgaaaacaatggtcgcgtcagatcacgtggcggtgatggggttacccaacccggggagtatgacggtggtggagtcgtgggtgatggtggagagcgtgacggaaacttgcattgaagatggagagatggagaagtgtacggacgtggagaaaatgaagaatctcgagaaggacacgtgtcccgggtttgtatcagatgggctgaaccgggttttctgggtgaaccaagcgtatagaaatatggtaggcttggaccaggacggggagcaagaacgggccgcaaccgccgtggggttggtgttgaaagacgggctcacctttccctgcggtgcattttcgtgccgcgtacggttgcgttacgtggatgggaagggtaagaattactcgaaaacggttccctgcgacatttggaagctgagctccggcggatttgcatggcggctggacgtcaacgctgctctcagcttgggactctag

SEQ ID NO: 27 - DNA, 849 bp, Artificial Sequence
  synthetic
atggaaggcagaggagggtgttgcatagcgagatatgcatcttctggccatggggtctatgatatgtcgaaagtgcaccggataatgctacggttccgaccgatcgctccgaaaccggcgacgggtggatcggtttccccacagggaggtagtgaattttgctctaagtcggggagaggaaaaagaagacactttaaagaaagtaacactatgagtaatacaaagaggtgcaacaagaaaagaagggttttgaacgaagagaagagggttactttgccacttctccctgaaacccctgactgcaaagaagagtctattctgaagcagcaaaaggatggagtagtccaaaagatgatgccgttttggttaagcttcggtgaagctggtgataaaaaggagggttttctcggtggtggtggtgtacggtcagcagatcaggcggtgtttatgggagggcagacggtgagagtggggtcgtgcgtgacggtggaatgcgtgacagagacatgggtgagtggggatgggctagggagtacagacgaggaaaagaaggtgaatctcgggagggacacgtgtccggggtttatttcggatgggttggggagggtaacgtggacaaatggggcgtataaggaaatggttggaggggaaacgatggtgtggttggtgatgaaggaaaggatgccgatgatgagaacgtacccggcgtttacatgcaaggtaagggtgcaatacacgtgcgggaaggagcggagttccttgacgctgccttgtgacgtttggcgaatggacggcggtggatttgcatggaggctcgatatcaacgctgctctttgcttggggcggtga

(19) United States
(12) Patent Application Publication
(10) Pub. No.: US 2019/0338304 A1
(43) Pub. Date: Nov. 7, 2019

(54) TRANSCRIPTION REPRESSOR GENE FAMILY AND USES THEREOF

(71) Applicant: Shucai WANG, Changchun (CN)

(72) Inventors: Shucai WANG, Changchun (CN); Hainan TIAN, Changchun (CN); Siyu CHEN, Changchun (CN); Tianya WANG, Changchun (CN); Kaijie ZHENG, Changchun (CN); Shanda LIU, Changchun (CN); Dongqiu LI, Changchun (CN)

(21) Appl. No.: 16/407,968

(22) Filed: May 9, 2019

Related U.S. Application Data
(63) Continuation of application No. PCT/CN2017/110375, filed on Nov. 10, 2017.

(30) Foreign Application Priority Data
Nov. 20, 2016 (CN) 201611032876.X

(51) Int. Cl. C12N 15/82; C07K 14/415; C12N 15/11; C12N 9/22

(52) U.S. Cl. CPC C12N 15/8271; C07K 14/415; C12N 2800/80; C12N 9/22; C12N 2310/20; C12N 15/11

(57) ABSTRACT

Disclosed are a family of abscisic acid (ABA)-induced transcription repressor genes AITRs and uses thereof for improving plant stress resistance. Mutants with knockout of AITRs genes in Arabidopsis thaliana exhibit enhanced resistance to ABA and abiotic stresses.

### CROSS-REFERENCE TO RELATED APPLICATIONS

This application is a continuation of International Patent Application No. PCT/CN2017/110375, filed on Nov. 10, 2017, which claims the benefit of priority from Chinese Application No. 201611032876.X, filed on Nov. 20, 2016. The content of the aforementioned application, including any intervening amendments thereto, is incorporated herein by reference.

### REFERENCE TO AN ELECTRONIC SEQUENCE LISTING

The contents of the electronic sequence listing (Untitled_ST25.txt; Size: 35,000 bytes; and Date of Creation: Jul. 22, 2019) is herein incorporated by reference in its entirety.

### TECHNICAL FIELD

The application relates to biotechnology, and more specifically to a family of transcription factor genes conserved in angiosperms, detection of transcriptional inhibitory activity of the proteins encoded by the family of transcription factor genes and their uses in the improvement for plant abiotic stress resistance. It was first proved in Arabidopsis thaliana that such genes encode a novel family of transcription repressors. Through amino acid sequence BLAST searching for homologues, the gene family is found to be widely presented in angiosperms. Plant abiotic stress resistance can be improved by knocking out such family genes in the plants, thus can be used for plant improvement.

### BACKGROUND

Environmental stresses, including abiotic stresses such as salinity, alkalinity, drought and chilling and biotic stresses such as pest and disease infection, affect plant growth and development, and are one of the most important constraints to agricultural and forestry production. Therefore, genetic modification to improve plant stress resistance is one of the most commonly used methods in modern plant molecular breeding. Abscisic acid (ABA) is one of the most important plant hormones that are involved in the regulation of several different aspects of plant growth and development such as seed germination, root development and senescence. Most importantly, ABA plays a key role in regulating plant response to abiotic stresses. Exogenous application of ABA can improve plant resistance to abiotic stresses. Based on the analysis of ABA signal transduction pathway, it has been demonstrated that several different proteins such as PYR/PYL/RCAC ABA receptors, PP2C protein phosphatases and SnRK2 protein kinases are involved in ABA signaling. Binding of ABA to the PYR/PYL/RCAC receptor proteins will promote their binding to the downstream PP2C proteins, thus inhibit the phosphatase activity of PP2Cs, resulting in the release and phosphorylation of SnRK2 kinases. The phosphorylated SnRK2 kinases can then phosphorylate downstream transcription factors, leading to the activation of the ABA response genes. Abiotic stresses such as salinity, alkalinity, drought and chilling induce ABA biosynthesis, leading to the activation of ABA signaling and induction of ABA-response genes.

So far, transcription factors from several different families including the MYB, NAC and bZIP families have been found to regulate ABA signaling, and overexpression of these transcription factor genes is able to enhance plant abiotic stress resistance, thus can be potentially used in plant molecular breeding. However, gene overexpression in the plants can only be achieved by generating genetically modified transgenic plants that are under regulation and control, thus greatly limiting the commercial applications of transcription factors known to participate in ABA signaling. Disclosed herein is a new gene family, ABA-induced transcription repressors (AITRs). The inventors demonstrated that AITRs in Arabidopsis thaliana are a family of transcription repressors, which is a plant-specific protein family as indicated by BLAST searching on the NCBI website. Further, homologues searching on the Phytozome website (see https://phytozome.jgi.doe.gov/pz/portal.html) indicates that AITRs are present in all genome sequenced angiosperms, and a total of 194 AITR genes are found in 45 species of angiosperms including rice (Oryza sativa), corn (Zea mays), sorghum (Sorghum bicolor), millet (Setaria italica), tomato (Solanum lycopersicum), potato (Solanum tuberosum), cucumber (Cucumis sativus), soybean (Glycine max) and rape (Brassica napus).

The analysis of AITRs from plants such as Arabidopsis, soybean and tomato has showed they all have transcriptional inhibitory activities, indicating that AITRs are a new and special family of transcriptional repressors in angiosperms, and participate in ABA signaling. Therefore, responses of plants to ABA and environmental stresses are affected by the overexpression or knockout of AITR genes.

### SUMMARY

The application provides a family of ABA-induced transcription repressor genes AITRs and uses thereof.

The AITRs are conserved in angiosperm.

The AITRs are selected from the group consisting of: evm_27.TU.AmTr_v1.0_scaffold00020.44, evm_27.TU.AmTr_v1.0_scaffold00065.128, GSMUA_AchrUn_randomG25040_001, GSMUAAchr8G28790_001, GSMUA Achr3G17030 001, GSMUA_Achr1G22520_001, Spipo2G0099700, Bradi4g39520, Bradi1g23180, Bradilg23171, Brast06G073500, Brast05G233600, 0s12g13910, 0s07g39700, Pahal.B04511, Pahal.J00645, Pavir.Ca00430, Pavir.J31265, Pavir.Bb01196, Pavir.Ba01082, Pavir.Ia02857, Seita.3G087100, Seita.2G371100, Seita.9G302900, Sevir.3G089200, Sevir.2G382000, Sobic.008G087500, Sobic.002G356300, GRMZM2G084005, GRMZM2G105302, GRMZM2G396402, Aquca_011_00277, Aquca_040_00014, Aquca_060_00028, Kalax .0359s0028, Kalax.0032s0036, Kalax.0157s0044, Kalax.0356s0038, Kalax.0893s0013, Kalax.0549s0038, Kalax.0574s0031, Kalax.0327s0041, Kalax.0008s0194, Kalax.0279s0003, Migut.D01238, Solyc03g111100, SolycO2g068030, Solyc02g093890, Solyc04g077740, PGSC0003DMG400020225, PGSC0003DMG400015196, Eucgr.I01669, Eucgr.B00140, Eucgr.I01670, GSVIVG01001833001, Lus10032077.g, Lus10014610.g, Lus10018671.g, Manes.17G106600, Manes.15G157400,

Manes.15G157500, Manes.17G106700, Manes.14G054200, Potri.001G334500, Potri.015G091900, Potri.012G094400, 30190.t010976, 30190.t010977, 30147.t013774, SapurV1A.0420s0190, SapurV1A.1554s0040, SapurV1A.0205s0110, SapurV1A.0004s0080, orange1.1g048740m.g, orange1.1g044567m.g, orange1.1 g020746m.g, Ciclev10016102m.g, Ciclev10021486m.g, Ciclev10015465m.g, evm.TU.supercontig_836.2, evm.TU.supercontig_836.3, evm.TU.supercontig_4.106, Gorai.006G039600, Gorai .011 G092600, Gorai.006G039500, Gorai.003G109600, Gorai.011G092700, Gorai.00IG061000, Thecc1EG017094, Thecc1EG017093, Thecc1EG014589, 484507, 947661, 947662, 496519, 949566, 485209, AT3G27250, AT5G40800, AT5G40790, AT5G50360, AT5G63350, AT3G48510, Bostr.0556s0401, Bostr.7200s0087, Bostr.7200s0086, Bostr.0568s0474, Bostr.18473s0099, Brara.F03221, Brara.B03281, Brara.D01097, Brara.A02381, Brara.B01593, Brara.D01096, Brara.F02152, Brara.F02225, Brara.B03843, Brara.I03014, Cagra.5650s0019, Cagra.10745s0005, Cagra.0248s0015, Cagra.10745s0006, Cagra.0888s0012, Cagra.0807s0002, Cagra.16111s0005, Carubv10017781m.g, Carubv10005474m.g, Carubv10007652m.g, Carubv10028617m.g, Carubv10027553m.g, Carubv10018515m.g, Carubv10011167m.g, Thhalv10004746m.g, Thhalv10028138m.g, Thha1v10028165m.g, Thhalv10010594m.g, Thhalv10015611m.g, Thha1v10005356m.g, Cucsa.136020, gene26388.1-v 1 .0-hybrid, gene06390.1-v1.0-hybrid, gene03749.1-v1.0-hybrid, gene26389.1-v1.0-hybrid, gene02593.1-v1.0-hybrid, Glyma.05 G045800, Glyma.17G127900, Glyma.04G193900, Glyma.06G172200, Glyma.05 G074200, Glyma.19G076500, MDP0000122076, MDP0000543092, MDP0000262573, MDP0000685813, MDP0000215071, MDP0000807011, MDP0000314792, Medtr6g023190, Medtr4g103840, Medtr3g075030, Phvu1.004G056400, Phvu1.003G209000, Phvu1.009G168000, Prupe.3G301600, Prupe.3G301500, Prupe.5G154700, Prupe.1G421700, BnaC01g25440D, BnaA02g11740D, BnaC02g16130D, BnaA02g28730D, BnaA02g33690D, BnaC02g42480D, BnaC02g48140D, BnaA04g10610D, BnaA04g10620D, BnaC04g32900D, BnaC04g32910D, BnaA06g18940D, BnaA06g32290D, BnaC07g24110D, BnaAnng12320D, CotAD_19897, CotAD_73582, CotAD_54468, CotAD_54469, CotAD_59800, CotAD_19898, CotAD_73583, CotAD_15612, CotAD_01868, CotAD_15613 and CotAD_15614.

Examples of the AITRs nucleotide sequence are shown in SEQ ID Nos. 1-17.

The application further provides a use of the family of the transcription repressor genes AITRs in generation of a plant with enhanced abiotic stress tolerance.

A method of improving resistance of a plant to abiotic stresses comprises:

inhibiting or knocking out the family of the transcription repressor genes in the plant.

The plant is angiosperm.

The knocking out is carried out by using a CRISPR/Cas9 gene editing technique.

The application has the following beneficial effects.

The present invention demonstrates that AITRs in Arabidopsis thaliana are a new family of transcription repressors, and a total of 194 homologous proteins are identified from 45 species of angiosperms using amino acid sequence for BLAST searching. Examination of AITRs in soybean, tomato, cotton and rape indicates that they all have transcription inhibitory activities, indicating that the family of transcription factors disclosed herein is special and conserved in angiosperms. The resistance of Arabidopsis plants to ABA and abiotic stresses is improved through the knockout of AITRs, and thus it can be concluded that the AITRs function as negative regulators in plant response to abiotic stresses. Therefore, plant resistance to abiotic stresses can be enhanced by knocking out AITRs.

Therefore, the present invention generally provides a family of transcription factor genes for genetic modification to improve plant resistance to abiotic stresses.

In order to identify the transcription factors involved in ABA signaling, the inventors obtain a large number of ABA-induced genes by using some existing genomic and microarray analysis data. By analyzing the corresponding proteins, it has been found that the amino acid sequence of one of the proteins contains a transcriptional repression motif L×L×L, which is conserved in the transcription repressors Aux/IAAs and ERFs. A total of 6 genes are found in Arabidopsis thaliana to encode similar proteins by using amino acid sequence for homologues searching and are named AITRI/At3g27250, AITR2/At3g48510, AITR3/At5g40790, AITR4/At5g40800, AITR5/At5g50360 and AITR6/At5g63350, respectively. Quantitative RT-PCR results show that expression of the six genes is induced by ABA. In addition, all the six AITRs are found to be present in the nucleus by transfection of Arabidopsis thaliana protoplasts, and can inhibit the expression of the reporter gene, demonstrating that AITRs is a novel family of ABA-induced transcription factor genes.

Full-length amino acid sequences of Araidopsis AITRs are used for BLAST searching on the NCBI website and the results indicate the absence of homologues in the species other than plants. This demonstrates that they are a plant-special transcription factor family. The full-length amino acid sequence of Arabidopsis AITRs is then used for BLAST searching for homologues in various plants on the website such as Phytozome, and the results indicate that the AITRs homologues are only found in angiosperms. Further, a total of 194 homologous protein-coding genes are identified in 45 species of plants including rice, corn, sorghum, millet, tomato, potato, cucumber, soybean and rape.

The expression of AITRs in soybean, tomato, cotton and rape is also induced by ABA as examined by RT-PCR. Transient transfection analysis using Arabidopsis protoplasts also confirms that AITRs in soybean, tomato, cotton and rape have transcriptional inhibitory activities, indicating that AITRs are a conserved family of transcription repressors in angiosperms.

The AITR1-AITR6 genes are cloned into the pZP211 binary expression vector, respectively, transformed into Agrobacterium GV3101 cells to transform Arabidopsis plants, and transgenic plants overexpressing AITR genes were obtained. T-DNA insertion mutant lines were obtained from TAIR, and 4 mutants with AITR genes knocked out are identified. The corresponding double and triple mutants were generated by crossing. By using the AITR overexpression plants and the knockout mutants, it is found that the sensitivity to ABA and abiotic stresses such as salinity or alkalinity is reduced in the knockout mutants, indicating that AITRs participate in ABA signaling and the plant resistance to abiotic stresses, and therefore plant resistance to abiotic stresses can be enhanced through the knockout of the AITR genes.

The application provides a novel and special family of transcription factors involving the regulation of ABA signal transduction and plant resistance to abiotic stresses. Plant resistance to abiotic stresses is improved through the knockout of the AITR genes using CRISPR/Cas9 techniques.

The present invention provides candidate genes that can be targeted for genome editing in molecular breeding, therefore to generate transgene-free mutant plants with enhanced tolerance to abiotic stresses.

### BRIEF DESCRIPTION OF THE DRAWINGS

FIGS. 1A-1B show the induction of the expression of AITR genes in Arabidopsis by ABA.

FIGS. 2A-2C show the identification of AITRs in Arabidopsis as a novel family of transcription repressors.

FIG. 3 shows the presence of AITR proteins only in angiosperms.

FIGS. 4A-4D show that AITRs in Glycine max and Solanum lycopersicum are ABA-induced transcription repressors.

FIG. 5 shows the reduction in response to ABA and abiotic stresses in the Arabidopsis AITR gene-knockout mutants.

FIGS. 6A-6F show that AITRs in Brassica napus and Gossypium hirsutum are ABA-induced transcription repressors.

FIG. 7 shows the construction of a transgene-free Solanum lycopersicum mutant through the knockout of SlAITR1 gene using CRISPR/Cas9 gene editing technology.

FIG. 8 shows the alignment of amino acid sequences of AITRs in Solanum lycopersicum.

FIG. 9 shows the alignment of amino acid sequences of AITRs in Glycine max.

FIG. 10 shows the alignment of amino acid sequences of AITRs in Brassica napus.

FIG. 11 shows the alignment of amino acid sequences of AITRs in Gossypium hirsutum.

### DETAILED DESCRIPTION OF EMBODIMENTS

### Example 1 Identification of Arabidopsis thaliana AITRs

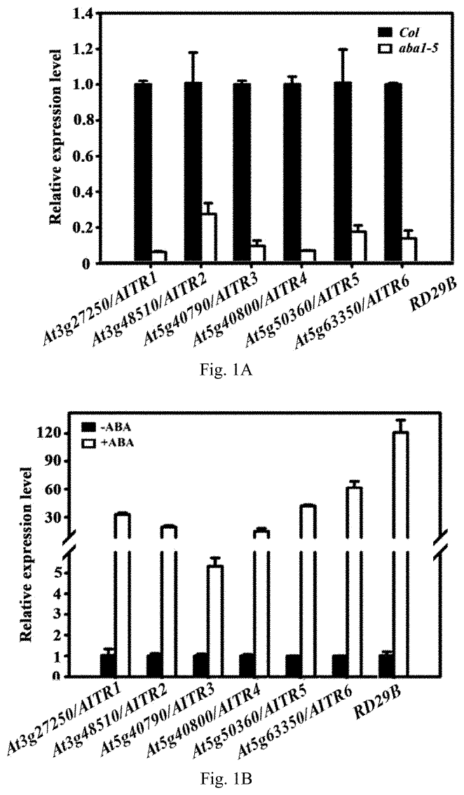

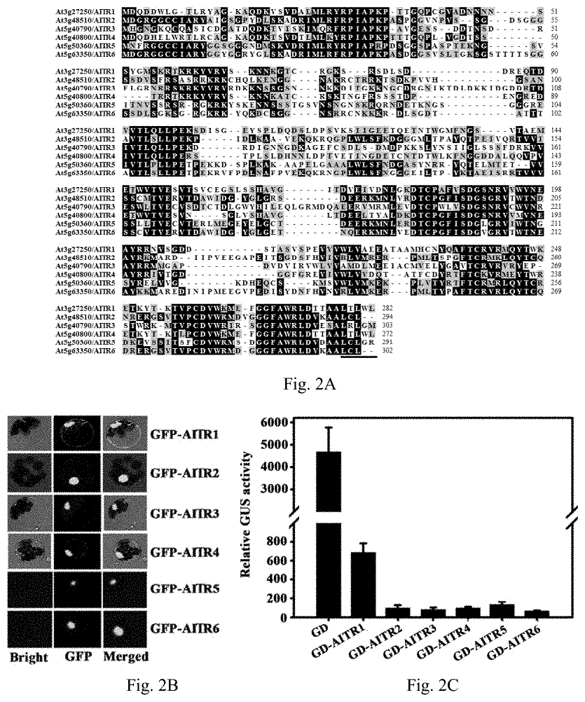

Based on the available transcriptome data, it has been found that the expressions of six closely related functionally unknown genes At3g27250, At3g48510, At5g40790, At5g40800, At5g50360 and At5g63350 are induced by ABA. The qRT-PCR results also showed that the expressions of these six genes can be induced by exogenous ABA treatment, and the expression levels of these genes were significantly reduced in an ABA-deficient mutant aba 1-5 (FIGS. 1A-1B). The amino acid sequence analysis revealed that the At3g27250-encoded proteins had a transcriptional repression motif L×L×L. Arabidopsis thaliana protoplast transfection assays demonstrated that all the proteins encoded by these six genes were located in the nucleus and can inhibit the expressions of the reporter gene. The proteins encoded by the six genes were named ABA-induced transcription repressor 1 (AITR1), AITR2, AITR3, AITR4, AITR5 and AITRG, respectively to reflect their characteristics (see FIGS. 2A-2C). The results indicated that AITRs are a new family of transcription factors.

### Example 2 Identification of AITRs from Different Plants

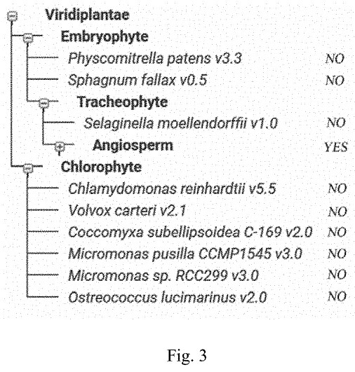

The full amino acid sequence of Arabidopsis AITR1 was used for homologues BLAST searching on the NCBI website (see https://blast.ncbi.nlm.nih.gov/Blast.cgi), and the results revealed that its homologous proteins were only present in plants. The full amino acid sequence of Arabidopsis AITR1 was then used for homologues BLAST searching for each plant on the phytozome website (see https://phytozome.jgi.doe.gov/pz/portal.html), and the results showed that homologous proteins were only present in angiosperms (FIG. 3). In addition, all angiosperms have homologous protein-encoding genes with a varying number, and a total of 188 AITR genes were found in 44 species of plants in addition to Arabidopsis thaliana. Information about the 194 AITR genes in 45 species of plants including Arabidopsis thaliana was shown in Table 1. The results indicated that AITRs are a family of conserved proteins in angiosperms.

TABLE 1
Distribution of AITRs in angiosperms

| | Number | | Amino |
|---|---|---|---|
| | of | | acid |
| Plant | genes | Locus name | number |
| Amborella | 2 | evm_27.TU.AmTr_v1.0_scaffold00020.44, | 340-383 |
| trichopoda | | evm_27.TU.AmTr_v1.0_scaffold00065.128 | |
| Musa acuminata | 4 | GSMUA_AchrUn_randomG25040_001, | 290-300 |
| | | GSMUA_Achr8G28790_001, | |
| | | GSMUA_Achr3G17030_001, | |
| | | GSMUA_Achr1G22520_001 | |
| Spirodela polyrhiza | 1 | Spipo2G0099700 | 346 |
| Brachypodium | 3 | Bradi4g39520, Bradi1g23180, Bradi1g23171 | 279-329 |
| distachyon | | | |
| Brachypodium | 2 | Brast06G073500, Brast05G233600 | 302-315 |
| stacei | | | |
| Oryza sativa | 2 | Os12g13910, Os07g39700 | 329-343 |
| Panicum hallii | 2 | Pahal.B04511, Pahal.J00645 | 317-423 |
| Panicum virgatum | 5 | Pavir.Ca00430, Pavir.J31265, Pavir.Bb01196, | 254-448 |
| | | Pavir.Ba01082, Pavir.Ia02857 | |
| Setaria italica | 3 | Seita.3G087100, Seita.2G371100, | 286-335 |
| | | Seita.9G302900 | |
| Setaria viridis | 2 | Sevir.3G089200, Sevir.2G382000 | 319-328 |
| Sorghum bicolor | 2 | Sobic.008G087500, Sobic.002G356300 | 325-334 |
| Zea mays | 3 | GRMZM2G084005, GRMZM2G105302, | 312-327 |
| | | GRMZM2G396402 | |
| Aquilegia coerulea | 3 | Aquca_011_00277, Aquca_040_00014, | 287-360 |
| | | Aquca_060_00028 | |
| Kalanchoe | 10 | Kalax.0359s0028, Kalax.0032s0036, | 276-316 |
| marnieriana | | Kalax.0157s0044, Kalax.0356s0038, | |
| | | Kalax.0893s0013, Kalax.0549s0038, | |
| | | Kalax.0574s0031, Kalax.0327s0041, | |
| | | Kalax.0008s0194, Kalax.0279s0003 | |
| Mimulus guttatus | 1 | Migut.D01238 | 310 |
| Solanum | 4 | Solyc03g111100, Solyc02g068030, | 275-310 |
| lycopersicum | | Solyc02g093890, Solyc04g077740 | |
| Solanum tuberosum | 2 | PGSC0003DMG400020225, | 282-309 |
| | | PGSC0003DMG400015196 | |
| Eucalyptus grandis | 3 | Eucgr.I01669, Eucgr.B00140, Eucgr.I01670 | 301-327 |
| Vitis vinifera | 1 | GSVIVG01001833001 | 268 |
| Linum | 3 | Lus10032077.g, Lus10014610.g, | 265-357 |
| usitatissimum | | Lus10018671.g | |
| Manihot esculenta | 5 | Manes.17G106600, Manes.15G157400, | 292-316 |
| | | Manes.15G157500, Manes.17G106700, | |
| | | Manes.14G054200 | |
| Populus | 3 | Potri.001G334500, Potri.015G091900, | 320-354 |
| trichocarpa | | Potri.012G094400 | |
| Ricinus communis | 3 | 30190.t010976, 30190.t010977, 30147.t013774 | 299-314 |
| Salix purpurea | 4 | SapurV1A.0420s0190, SapurV1A.1554s0040, | 319-365 |
| | | SapurV1A.0205s0110, SapurV1A.0004s0080 | |
| Citrus sinensis | 3 | orange1.1g048740m.g, orange1.1g044567m.g, | 288-322 |
| | | orange1.1g020746m.g | |
| Citrus clementina | 3 | Ciclev10016102m.g, Ciclev10021486m.g, | 288-404 |
| | | Ciclev10015465m.g | |
| Carica papaya | 3 | evm.TU.supercontig_836.2, | 294-318 |
| | | evm.TU.supercontig_836.3, | |
| | | evm.TU.supercontig_4.106 | |
| Gossypium | 6 | Gorai.006G039600, Gorai.011G092600, | 279-300 |
| raimondii | | Gorai.006G039500, Gorai.003G109600, | |
| | | Gorai.011G092700, Gorai.001G061000 | |
| Theobroma cacao | 3 | Thecc1EG017094, Thecc1EG017093, | 295-341 |
| | | Thecc1EG014589 | |
| Arabidopsis lyrata | 6 | 484507, 947661, 947662, 496519, 949566, | 269-304 |
| | | 485209 | |
| Arabidopsis | 6 | AT3G27250, AT5G40800, AT5G40790, | 272-303 |
| Thaliana | | AT5G50360, AT5G63350, AT3G48510 | |
| Boechera stricta | 6 | Bostr.0556s0401, Bostr.7200s0087, | 272-309 |
| | | Bostr.7200s0086, Bostr.0568s0474, | |
| | | Bostr.18473s0099, Bostr.15774s0183 | |
| Brassica rapa | 10 | Brara.F03221, Brara.B03281, Brara.D01097, | 288-358 |
| | | Brara.A02381, Brara.B01593, Brara.D01096, | |
| | | Brara.F02152, Brara.F02225, Brara.B03843, | |
| | | Brara.I03014 | |
| Capsella | 7 | Cagra.5650s0019, Cagra.10745s0005, | 274-362 |
| grandiflora | | Cagra.0248s0015, Cagra.10745s0006, | |
| | | Cagra.0888s0012, Cagra.0807s0002, | |
| | | Cagra.16111s0005 | |
| Capsella rubella | 7 | Carubv10017781m.g, Carubv10005474m.g, | 274-362 |
| | | Carubv10007652m.g, Carubvl0028617m.g, | |
| | | Carubv10027553m.g, Carubvl0018515m.g, | |
| | | Carubv10011167m.g | |
| Eutrema | 6 | Thhalv10004746m.g, Thhalv10028138m.g, | 279-296 |
| salsugineum | | Thhalv10028165m.g, Thhalv10010594m.g, | |
| | | Thhalv10015611m.g, Thhalv10005356m.g | |
| Cucumis sativus | 1 | Cucsa.136020 | 315 |
| Fragaria vesca | 5 | gene26388.1-v1.0-hybrid, gene06390.1-v1.0- | 272-376 |
| | | hybrid, gene03749.1-v1.0-hybrid, gene26389.1- | |
| | | v1.0-hybrid, gene02593.1-v1.0-hybrid | |
| Glycine max | 6 | Glyma.05G045800, Glyma.17G127900, | 246-309 |
| | | Glyma.04G193900, Glyma.06G172200, | |
| | | G1yma.05G074200, Glyma.19G076500 | |
| Malus domestica | 7 | MDP0000122076, MDP0000543092, | 242-703 |
| | | MDP0000262573, MDP0000685813, | |
| | | MDP0000215071, MDP0000807011 | |
| | | MDP0000314792 | |
| Medicago | 3 | Medtr6g023190, Medtr4g103840, | 236-292 |
| truncatula | | Medtr3g075030 | |
| Phaseolus vulgaris | 3 | Phvul.004G056400, Phvul.003G209000, | 262-300 |
| | | Phvul.009G168000 | |
| Prunus persica | 4 | Prupe.3G301600, Prupe.3G301500, | 295-377 |
| | | Prupe.5G154700, Prupe.1G421700 | |
| Brassica napus | 15 | BnaC01g25440D, BnaA02g11740D, | 258-485 |
| | | BnaC02g16130D, BnaA02g28730D, | |
| | | BnaA02g33690D, BnaC02g42480D, | |
| | | BnaC02g48140D, BnaA04g10610D, | |
| | | BnaA04g10620D, BnaC04g32900D, | |
| | | BnaC04g32910D, BnaA06g18940D, | |
| | | BnaA06g32290D, BnaC07g24110D, | |
| | | BnaAnng12320D | |
| Gossypium | 11 | CotAD_19897, CotAD_73582, CotAD_54468, | 130-300 |
| hirsutum | | CotAD_54469, CotAD_59800, CotAD_19898, | |
| | | CotAD_73583, CotAD_15612, CotAD_01868, | |
| | | CotAD_15613, CotAD_15614 | |

### Example 3 Detection of Transcriptional Activity of AITRs in Glycine max and Solanum lycopersicum

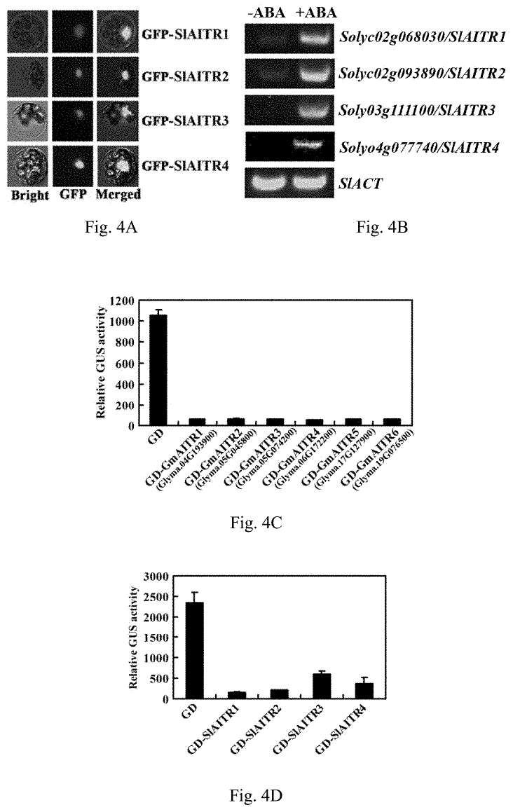

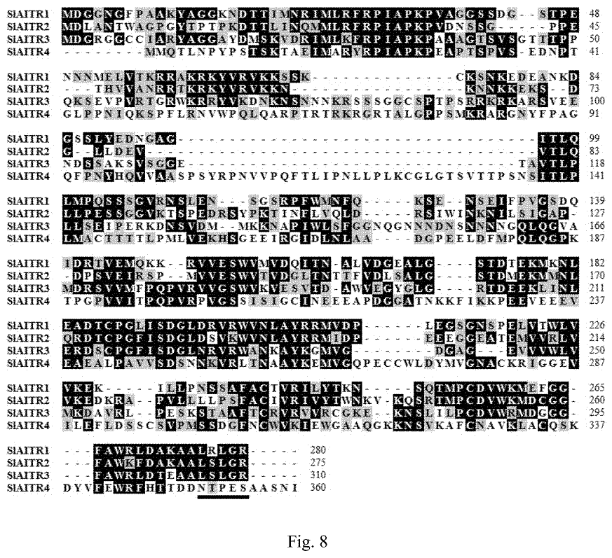

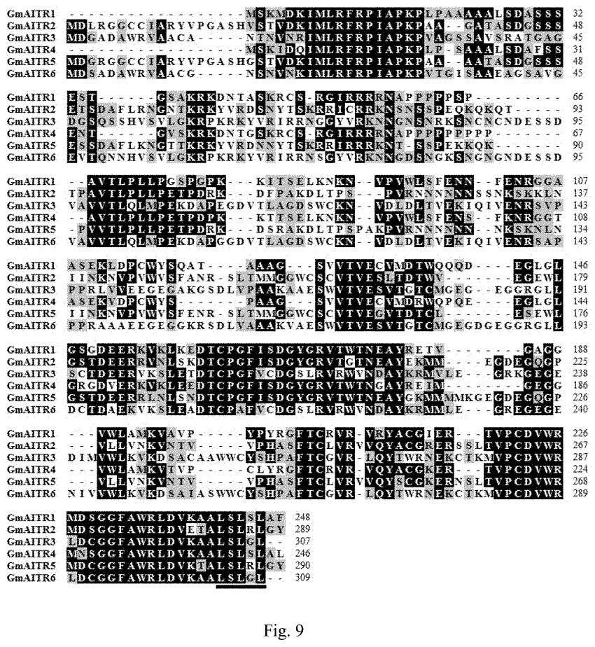

Solanum lycopersicum was used as an example to examine whether the AITR genes in other plants were also induced by ABA, and the results revealed that expressions of the four AITR genes in Solanum lycopersicum were induced by ABA. Protoplast transfection results showed that the four AITRs in Solanum lycopersicum were also located in the nucleus and inhibited the expressions of the reporter gene. Similarly, six AITRs in Glycine max were also transcription repressors (FIGS. 4A-4D, 8 and 9). The results indicated that AITRs are a new family of transcription repressors conserved in angiosperms.

### Example 4 Role of AITRs in ABA Signal Transduction and Plant Stress Resistance in Arabidopsis thaliana

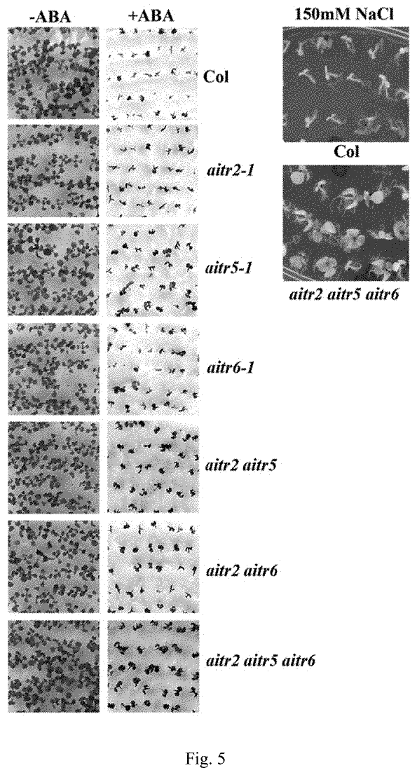

To examine the function of the AITR genes, transgenic plants overexpressing each of the six Arabidopsis thaliana AITR genes were generated, and knockout mutants for four of the genes were identified from purchased T-DNA insertion lines. The knockout mutants were then used to generate double and triple mutants by crossing. Researches on these transgenic plants and mutants revealed that the sensitivity of the mutants to ABA and abiotic stresses was reduced (FIG. 5). These results indicated that knockout of AITR genes improved of plant resistance to abiotic stresses.

### Example 5 Detection of Transcriptional Activity of AITRs in Brassica napus and Gossypium hirsutum

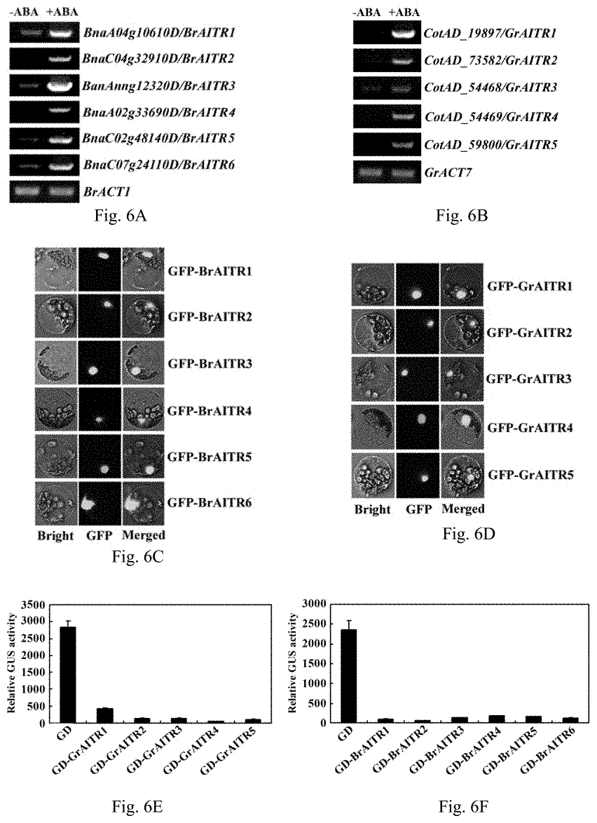

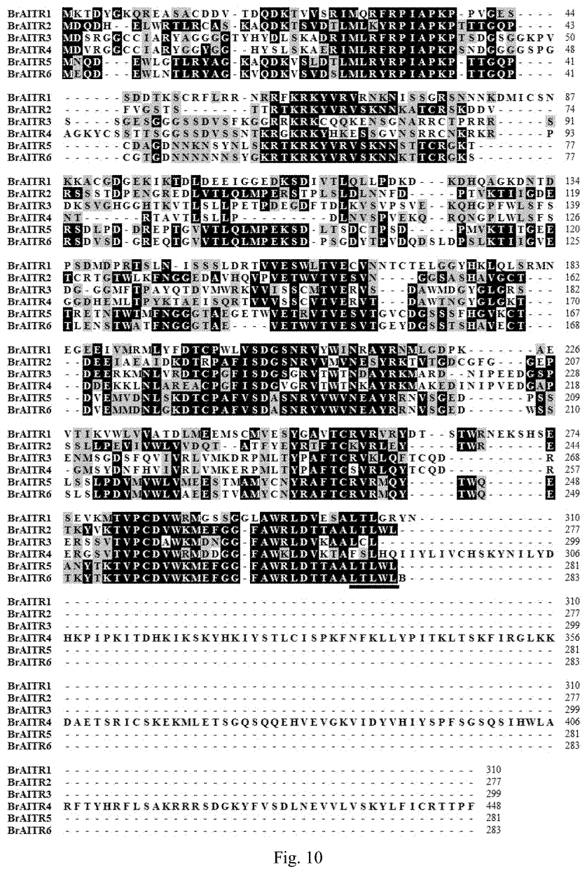

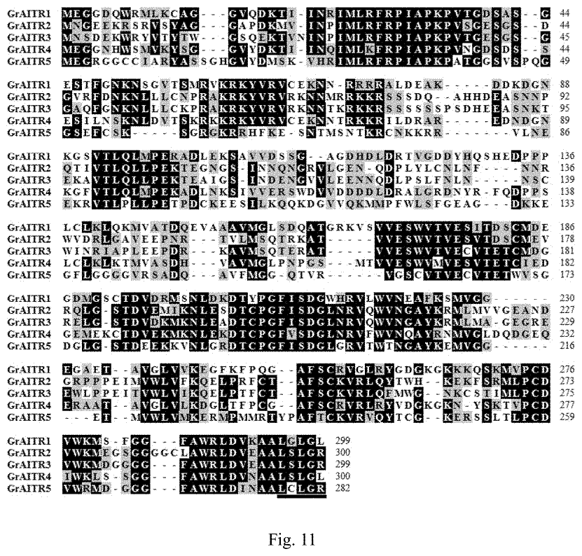

Brassica napus and Gossypium hirsutum were used as examples to further examine whether the AITR genes in other plants were also induced by ABA, and the results indicated that the expressions of six Brassica napus AITR genes and five Gossypium hirsutum AITR genes were induced by ABA. The protoplast transfection results showed that the six Brassica napus AITRs and five Gossypium hirsutum AITRs were all localized in the nucleus and could inhibit the expressions of the reporter gene (FIGS. 6A-6F, 10 and 11). The results indicated that AITRs are a new family of transcription repressors conserved in angiosperms.

Example 6 Generation of Transgene-Free Solanum lycopersicum Mutant by Knockout of SlAITR1 Gene Using CRISP1R/Cas9 Gene Editing

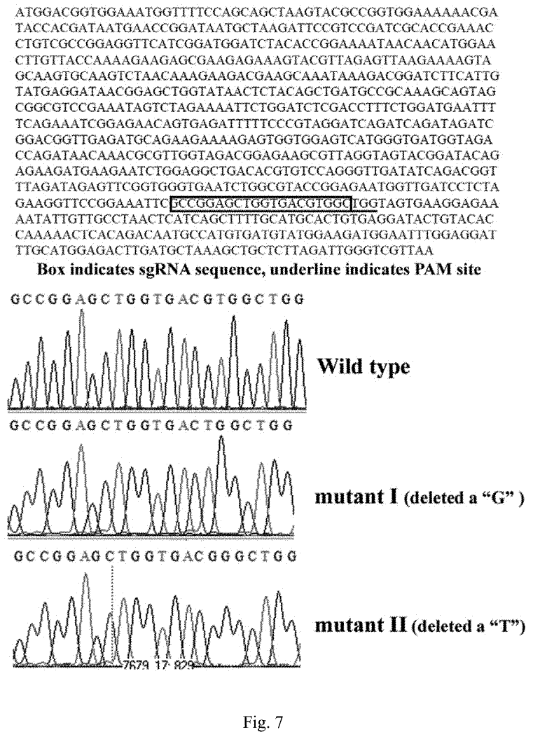

Knockout of AITR gene SlAITR1 in Solanum lycopersicum was used as an example to determine whether it is feasible to generate transgene-free mutants with AITR genes knocked out by using gene editing techniques such as CRISPR/Cas9. First, the gene sequence was obtained as follows. The locus number “Solyc02g068030” corresponding to SlAITR1 was used as a key word to search the Solanum lycopersicum genome through “Keyword search” on the Phytozome website (see https://phytozome.jgi.doe.gov/pz/portal.html), thereby obtaining the SlAITR1 gene sequence. Then, the target sequence was selected as follows. The sgRNA sequences for gene editing were predicated on the Crisprscan website (see http://www.crisprscan.org/?page=sequence) and the specificity of the obtained sequences was evaluated on the Cas-OFFinder website (see http://www.rgenome.net/cas-offinder/). A high specific sequence “GCCGGAGCTGGTGACGTGGC” was selected for gene editing. After that, construction of vectors, transformation of plants and examination of gene editing were performed as follows. The selected sgRNA was cloned into the CRISPR/Cas9 vector pHDE (https://www.addgene.org/789311) via PCR amplification and restriction enzyme digestion and ligation, and the construct was used for plant transformation. DNA of the transgenic plants was isolated and used for PCR amplification and sequencing to examine the editing status of SlAIITR1 gene and to isolate homozygous knockout mutants (see FIG. 7). Finally, Cas9 in progenies of the homozygous plants was examined by PCR to identify plants without Cas9 T-DNA insertion, i.e., transgene-free Solanum lycopersicum plants with SlAIITR1 gene knocked out. The results indicated that the gene editing technology such as CRISPR/Cas9 can be used to knock out the AITR genes in plants, thereby generating transgene-free mutant plants.

### INDUSTRIAL APPLICABILITY

The present invention provides a novel and special family of transcription factors that negatively regulating ABA signal transduction and plant resistance to abiotic stresses. Therefore, the plant resistance to abiotic stresses can be improved through the knockout of the related AITR genes using CRISPR/Cas9 techniques.

The application provides the candidate genes which can be edited for molecular breeding, and thus can be used to generate transgene-free plants with enhanced tolerance to abiotic stresses.

What is claimed is:

1. A method for improving plant resistance to abiotic stresses, comprising:
inhibiting or knocking out a family of transcription repressor genes AITRs in a plant.

2. The method of claim 1, Some of the AITRs have a nucleotide sequence as shown in SEQ ID Nos. 1-17.

3. The method of claim 1, wherein the AITRs are selected from the group consisting of evm_27.TU.AmTr_v1.0_scaffold00020.44, evm_27.TU.AmTr_v1.0_scaffold00065.128, GSMUA_AchrUn_randomG25040_001, GSMUA_Achr8G28790 001, GSMUA_Achr3G17030 001, GSMUA_Achr1G22520_001, Spipo2G0099700, Bradi4g39520, Bradi1g23180, Bradi1g23171, Brast06G073500, Brast05G233600, 0s12g13910, 0s07g39700, Pahal.B04511, Pahal.J00645, Pavir.Ca00430, Pavir.J31265, Pavir.Bb01196, Pavir.Ba01082, Pavir.Ia02857, Seita.3G087100, Seita.2G371100, Seita.9G302900, Sevir.3G089200, Sevir.2G382000, Sobic.008G087500, Sobic.002G356300, GRMZM2G084005, GRMZM2G105302, GRMZM2G396402, Aquca_011_00277, Aquca_040_00014, Aquca_060_00028, Kalax.0359s0028, Kalax.0032s0036, Kalax.0157s0044, Kalax.0356s0038, Kalax.0893s0013, Kalax.0549s0038, Kalax.0574s0031, Kalax.0327s0041, Kalax.0008s0194, Kalax.0279s0003, Migut.D01238, Solyc03g111100, Solyc02g068030, Solyc02g093890, Solyc04g077740, PGSC0003DMG400020225, PGSC0003DMG400015196, Eucgr.I01669, Eucgr.B00140, Eucgr.I01670, GSVIVG01001833001, Lus10032077.g, Lus10014610.g, Lus10018671.g, Manes.17G106600, Manes.15G157400, Manes.15G157500, Manes.17G106700, Manes.14G054200, Potri.001G334500, Potri.015G091900, Potri.012G094400, 30190.t010976, 30190.t010977, 30147.t013774, SapurV1A.0420s0190, SapurV1A.1554s0040, SapurV1A.0205s0110, SapurV1A.0004s0080, orange1.1g048740m.g, orange1.1g044567m.g, orange1.1g020746m.g, Ciclev10016102m.g, Ciclev10021486m.g, Ciclev10015465m.g, evm.TU.supercontig_836.2, evm.TU.supercontig_836.3, evm.TU.supercontig_4.106, Gorai.006G039600, Gorai.011G092600, Gorai.006G039500, Gorai.003G109600, Gorai.011G092700, Gorai.001G061000, Thecc1EG017094, Thecc1EG017093, Thecc1EG014589, 484507, 947661, 947662, 496519, 949566, 485209, AT3G27250, AT5G40800, AT5G40790, AT5G50360, AT5G63350, AT3G48510, Bostr.0556s0401, Bostr.7200s0087, Bostr.7200s0086, Bostr.0568s0474, Bostr.18473s0099, Brara.F03221, Brara.B03281, Brara.D01097, Brara.A02381, Brara.B01593, Brara.D01096, Brara.F02152, Brara.F02225, Brara.B03843, Brara.I03014, Cagra.5650s0019, Cagra.10745s0005, Cagra.0248s0015, Cagra.10745s0006, Cagra.0888s0012, Cagra.0807s0002, Cagra.16111s0005, Carubv10017781m.g, Carubv10005474m.g, Carubv10007652m.g, Carubv10028617m.g, Carubv10027553m.g, Carubv10018515m.g, Carubv10011167m.g, Thhalv10004746m.g, Thhalv10028138m.g, Thha1v10028165m.g, Thha1v10010594m.g, Thhalv10015611m.g, Thha1v10005356m.g, Cucsa.136020, gene26388.1-v1.0-hybrid, gene06390.1-v1.0-hybrid, gene03749.1-v1.0-hybrid, gene26389.1-v1.0-hybrid, gene02593.1-v1.0-hybrid, Glyma.05G045800, Glyma.17G127900, Glyma.04G193900, Glyma.06G172200, Glyma.05G074200, Glyma.19G076500, MDP0000122076, MDP0000543092, MDP0000262573, MDP0000685813, MDP0000215071, MDP0000807011, MDP0000314792, Medtr6g023190, Medtr4g103840, Medtr3g075030, Phvu1.004G056400, Phvu1.003G209000, Phvu1.009G168000, Prupe.3G301600, Prupe.3G301500, Prupe.5G154700, Prupe.1G421700, BnaC01g25440D, BnaA02g11740D, BnaC02g1613 OD, BnaA02g28730D, BnaA02g33690D, BnaC02g42480D, BnaC02g48140D, BnaA04g10610D, BnaA04g10620D, BnaC04g32900D, BnaC04g32910D, BnaA06g18940D, BnaA06g32290D, BnaC07g24110D, BnaAnng12320D, CotAD_19897, CotAD_73582, CotAD_54468, CotAD_54469, CotAD_59800, CotAD_19898, CotAD_73583, CotAD_15612, CotAD_01868, CotAD_15613 and CotAD 15614.

4. The method of claim 1, wherein the plant is angiosperm.

5. The method of claim 4, wherein the plant is Oryza sativa, Zea mays, Sorghum bicolor, Setaria italica, Solanum lycopersicum, Solanum tuberosum, Cucumis sativus, Glycine max or Brassica napus.

6. The method of claim 5, wherein the plant is Solanum lycopersicum.

7. The method of claim 1, wherein the knocking out is carried out by using a CRISPR/Cas9 gene editing technique.

8. The method of claim 1, wherein the abiotic stresses are caused by drought and/or salinity or alkalinity or other abiotic stresses.
